(19) United States
(12) Patent Application Publication
(10) Pub. No.: US 2008/0275502 A1
(43) Pub. Date: Nov. 6, 2008

(54) Lifting instrument for removing skinfolds and plasty method of removing skinfolds using the same

(76) Inventor: Jae-Young Park, Seoul (KR)

Correspondence Address: ARENT FOX KINTNER PLOTKIN & KAHN, PLLC;Suite 400, 1050 Connecticut Avenue, N.W., Washington, DC, 20036-5339, US

(21) Appl. No.: 11/797,134

(22) Filed: May 1, 2007

(51) Int. Cl. A61B 17/00

(52) U.S. Cl. 606/228; 128/898

(57) ABSTRACT

Disclosed therein are lifting instrument and method of removing skinfolds. The lifting instrument for removing skinfolds includes: a Gore-tex thread with a thickness ranging from 1.0 mm to 2.3 mm used for lifting skin tissues after being combined to the skin tissues; and needles disposed on at least one side of the Gore-tex thread, each needle having an arrow-like fluent curve and a straight line length ranging from 40 mm to 140 mm.

The lifting instrument and method can be applied to any body region through an easy and simple surgery, can achieve the same surgery result for each surgery regardless of a doctor's experiences or skills so that the doctor and a patient are all satisfied with the surgery result, can keep the satisfactory surgery result longer than the prior arts, and can ensure that the patient rapidly returns to his or her normal life with a rapid recovery after the surgery.

### BACKGROUND OF THE INVENTION

1. Field of the Invention

The present invention relates to lifting instrument for removing skinfolds and plasty method of removing skinfolds using the same, and more particularly, to lifting instrument for removing skinfolds and plasty method of removing skinfolds using the same, which can be applied to any body region through an easy and simple surgery, can achieve the same surgery result for each surgery regardless of a doctor's experiences or skills so that the doctor and a patient are all satisfied with the surgery result, can keep the satisfactory surgery result longer than the prior arts, and can ensure that the patient rapidly returns to his or her normal life with a rapid recovery after the surgery.

2. Background Art

In general, a plastic surgery is a general term of a surgical operation carried out for the sake of correction or beauty treatment of a partially damaged region or a malformed region of the human body, but, recently, most of the plastic surgeries are carried out for the purpose of beauty treatment.

According to a research, it has been reported that most of patients who undergoes the plastic surgery are positive and realistic persons who want to make a social life with self-confidence by correcting deficient regions of their bodies to thereby obtain and achieve better results. Moreover, to correct an abnormal figure into a normal figure or to make efforts to have a harmonized beauty is the human nature to make a healthy and rich life.

The plastic surgery is performed for various purposes, but particularly, performed to restore a corresponding wrinkled body region tightly in case where the skin of a specific region is drooped or a physical figure is changed into an ill appearance due to a fat accumulation, namely, in case where the specific region becomes folded.

Plastic surgery methods for removing skinfolds are classified into an invasive surgery using a method of removing drooped tissues after largely excising the skin and a non-invasive surgery for removing skinfolds by lifting the wrinkled skin after simply hanging a thread on the appropriate region without excision of the skin.

Of course, in the former case, since the skin needs to be excessively excised, the plastic surgery is very difficult and it takes much time for a patient to be completely recovered after the surgery.

So, recently, the latter surgery method has been widely used. The latter surgery method is also called APTOS.

APTOS is to remove skinfolds and tighten the skin by lifting up the aged skin and aged soft tissues inside the skin together using a specially-treated thread, for instance, a thread of 0.5 mm in diameter, which is combined with cogs or made of synthetic resin such as silicon or polypropylene, without excision of the skin, whereby APTOS can minimize a scar, which is the greatest demerit of the invasive surgery, reduce a stimulus or a damage of the operated portion and bleeding or swelling generated by the surgery, and provide a natural tension after the surgery to make the skin elastic and young since needles are used together with a simple local anesthesia in APTOS.

However, as described above, when the thread of 0.5 mm in diameter, which is made of synthetic resin, is used, if an excessive tension is applied to the thread, tissues are torn or a sawing or eating phenomenon occurs. Particularly, the typical threads according to the prior arts have a problem in that the lifting effect is considerably deteriorated as time goes by (within about six months).

Particularly, the typical thread simply combined with the cogs provides a good effect at the early days after the surgery, but still entails a problem in that the lifting effect is greatly deteriorated since the cogs slide naturally over time (within about six months).

Therefore, the method of removing skinfolds using the typical thread cannot provide a satisfactory surgery result to both the doctor and the patient since the surgery result is varied according to the doctors' skills. In addition, the method has several demerits in that the surgery effect cannot be kept long and a postoperative recovery is slow.

### SUMMARY OF THE INVENTION

Accordingly, the present invention has been made to solve the above-mentioned problems occurring in the prior arts, and it is an object of the present invention to provide lifting instrument for removing skinfolds and plasty method of removing skinfolds using the same, and more particularly, to lifting instrument for removing skinfolds and plasty method of removing skinfolds using the same, which can be applied to any body region through an easy and simple surgery, can achieve the same surgery result for each surgery regardless of a doctor's experiences or skills so that the doctor and a patient are all satisfied with the surgery result, can keep the satisfactory surgery result longer than the prior arts, and can ensure that the patient rapidly returns to his or her normal life with a rapid recovery after the surgery.

To accomplish the above object, according to the present invention, there is provided a lifting instrument for removing skinfolds comprising: a Gore-tex thread combined to a predetermined skin tissue to substantially lift up the skin tissue; and a needle disposed on at least one of both ends of the Gore-tex thread.

Here, the Gore-tex thread has a thickness ranging from 1.0 mm to 2.3 mm. In this instance, if the thickness of the Gore-tex thread is less than 1.0 mm, the sawing or eating phenomenon occurs or the thread is broken when excessive tension is applied to the thread, but if the thickness of the Gore-tex thread is more than 2.3 mm, an operated portion of a patient's body may be unnatural, and so, it is preferable that the thickness of the Gore-tex thread ranges from 1.0 mm to 2.3 mm.

In addition, it has been reported that Gore-tex provides good biocompatibility to the human body and has little side effects inside the human body differently form silicon. Moreover, it has been verified that Gore-tex is a smooth and soft material, and so, is the best compatible material to be used in a plastic surgery for removing skinfolds since it does not make the operated portion unnatural and provide a foreign body sense even though the thickness of the Gore-tex thread is about 2.0 mm.

Meanwhile, a needle, which has an arrow-like curve and a straight line length ranging from 40 mm to 140 mm, is combined to at least one end of the Gore-tex thread. But, for convenience of the surgery, the needles are combined to both ends of the Gore-tex thread, and removed from the thread after the surgery.

The needle is used to insert the Gore-tex thread to a space under the corium by pricking the corium located under the cuticle, and its form and length are varied according to the operated portion. However, it has been reported that the needle with the arrow-like curve is the most convenient in use since the needle is vertically inserted to the corium and horizontally inserted long in the space under the corium. Moreover, it has been verified that the straight line length ranging from 40 mm to 140 mm is the most proper in use in consideration of the thickness and form of the corium tissues of the face, the breast, the hip, the abdomen and the vagina, which are a target region in the plastic surgery.

Meanwhile, the curvature radius of the needle can be varied according to the target region, and the needles combined to both ends of the Gore-tex thread may have the same curvature radius or different curvature radiuses.

For instance, in case of a lifting instrument for removing facial skinfolds, it is preferable that the Gore-tex thread has a thickness ranging from 1.0 mm to 1.5 mm, and one of the needles located at both sides of the Gore-tex thread has a straight line length ranging from 50 mm to 90 mm and the other one has a straight line length ranging from 40 mm to 60 mm.

In case of a lifting instrument for removing body skinfolds, it is preferable that the Gore-tex thread has a thickness ranging from 1.2 mm to 1.7 mm, and the needles located at both sides of the Gore-tex thread have a straight line length ranging from 100 mm to 140 mm.

In case of a lifting instrument for abdomen lifting, it is preferable that the Gore-tex thread has a thickness ranging from 1.8 mm to 2.3 mm, and the needles located at both sides of the Gore-tex thread have a straight line length ranging from 80 mm to 120 mm.

In case of a lifting instrument for vagina lifting, it is preferable that the Gore-tex thread has a thickness ranging from 1.0 mm to 1.5 mm, and the needles located at both sides of the Gore-tex thread have a straight line length ranging from 40 mm to 60 mm.

As described above, the ranges of the thickness of the thread and the length of the needles are determined in consideration of a depth of the operated portion to the corium layer and a range of the surgical operation. The thin thread (as thin as the tissues are not torn when tension is applied thereto) must be used to the face or the vagina region since the corium layer is not deep, but the thick thread (as thick as the thread is not felt or does not provide a foreign body sense inside the tissues) must be used to the body region (the breast and the hips) and the abdominal region, and so, the ranges of the thickness of the threads are determined as the above.

In addition, the short needles can be used to the vagina region since the operated area (a straight line length that the thread is inserted into the corium layer) is not long, but the long needles must be used to the body region (the breast and the hips) and the abdominal region since the operated area (a straight line length that the thread is inserted into the corium layer) is still longer. In addition, one of the needles to the face region is short and the other is long since the operated area (a straight line length that the thread is inserted into the corium layer) is long but the corium layer is not thick.

Meanwhile, there is no need to restrict the length of the thread since a sufficient length can be used according to the operated area (a straight line length that the thread is inserted into the corium layer), but it is preferable to use the thread with the length ranging from 100 cm to 120 cm in case of the lifting instrument for removing facial skinfolds, the thread with the length ranging from 70 cm to 90 cm in case of the lifting instrument for removing body skinfolds, the thread with the length ranging from 110 cm to 130 cm in case of the lifting instrument for the abdomen lifting, and the thread with the length ranging from 35 cm to 55 cm in case of the lifting instrument for the vagina lifting.

Meanwhile, to accomplish the above object, according to the present invention, there is provided a plasty method of removing skinfolds comprising the steps of: vertically forming a temporal excision to the horny integument, the multilayered epidermis and the corium layer under the epidermis at the target region where skinfolds will be removed; fixing an anchor at the temporal excision to form a fiducial point of tension; and hanging an end of the Gore-tex thread, which has a thickness ranging from 1.0 mm to 2.3 mm, on the anchor, and leading the other end of the Gore-tex thread to the target region through the corium under the epidermis, fixing the end of the thread to the fascia tissue of the drooped skin, and pulling the fixed thread end tightly.

Here, the Gore-tex thread is mounted not in a single-line type but in a two-line type to transmit power stably.

So, the Gore-tex thread 110 is put around in a form proper to the target region and fixed in such a way that both ends of the thread meet with each other or are adjacent to each other, so that the entire form of the Gore-tex thread does not become the single-line type but becomes the two-line type where the both ends of the thread are connected with each other.

As described above, since the Gore-tex thread is constructed not in the single-line type but in the two-line type, power is applied to both sides uniformly when the thread is pulled to thereby prevent that the thread is broken later due to an excessive extension at one side or that an unsatisfied result is obtained due to lengthening of a unextended side.

As described above, the Gore-tex thread is constructed in the two-line type, and according to circumstances, two strands of the thread may cross with each other at the center thereof once or more.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a view of a lifting instrument for removing facial skinfolds according to a first preferred embodiment of the present invention, wherein

FIG. 1a is a schematic structural diagram for showing a cheek lifting method,

FIG. 1b is a schematic structural diagram for showing a neck lifting method, and

FIG. 1c is a schematic structural diagram for showing a forehead lifting method;

FIG. 2 is a view of a lifting instrument for removing body skinfolds according to a second preferred embodiment of the present invention, wherein

FIG. 2a is a schematic structural diagram for showing a breast lifting method,

FIG. 2b is a schematic structural diagram for showing a hip lifting method,

FIG. 2c is a schematic structural diagram for showing an abdomen lifting method, and

FIG. 2d is a view of a lifting instrument for removing abdomen skinfolds for the abdomen lifting; and

FIG. 3 is a view of a lifting instrument for removing vagina rugae according to a second preferred embodiment of the present invention, wherein, wherein

FIG. 3a is a schematic structural diagram for showing a vagina lifting method.

### DETAILED DESCRIPTION OF THE PREFERRED EMBODIMENT

Reference will be now made in detail to the preferred embodiment of the present invention with reference to the attached drawings. In the drawings, the same reference numerals designate the same parts.

Here, a method of removing skinfolds will be described in brief. In fact, the method of removing skinfolds according to the present invention is a process to lift up the fascia tissues of the drooped skin using a Gore-tex thread, which will be described later.

The method of removing skinfolds according to the present invention includes the steps of: forming a temporal excision at a target region of the skin unexcessively; fixing an anchor at the target region through the temporal excision to form a fiducial point of tension; hanging an end of the Gore-tex thread, which has a thickness ranging from 1.0 mm to 2.3 mm, on the anchor; fixing and supporting the other end of the Gore-tex thread on the fascia tissue (hard tissue) of the drooped skin; and pulling the Gore-tex thread tightly.

Particularly, the method of removing skinfolds according to the present invention is applicable to all tissues of the human body, which can be drooped due to gravity, for instance, all regions of the face, the body and the vagina.

Now, referring to the drawings, plastic surgery methods to each appropriate region of the face, the body and the vagina will be described in detail.

Removal of Facial Skinfolds

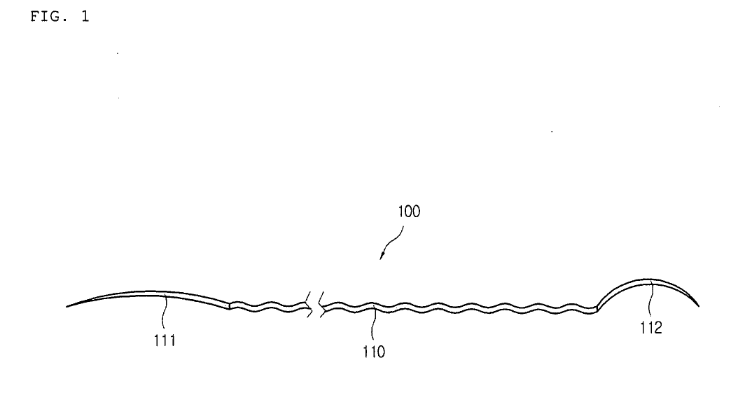

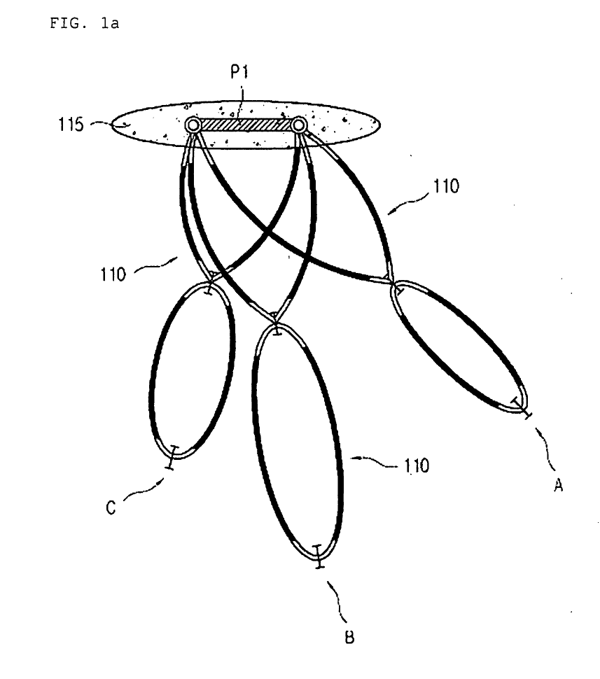

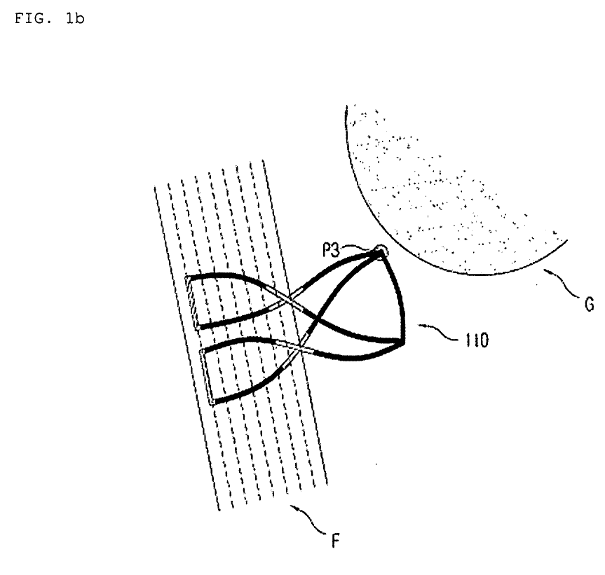

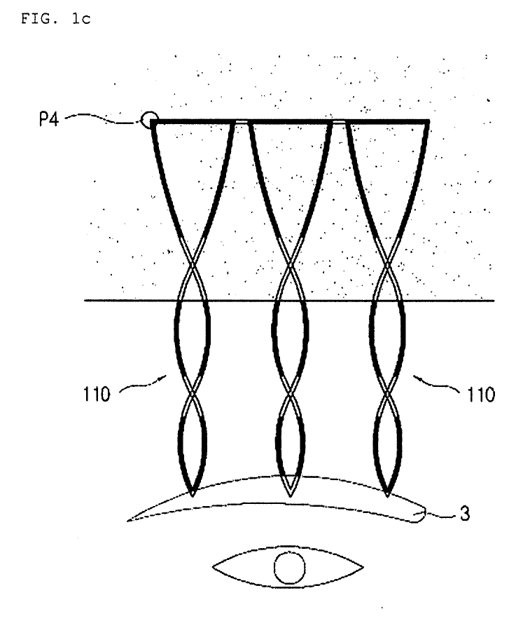

FIG. 1 is a view of a lifting instrument for removing facial skinfolds according to a first preferred embodiment of the present invention, wherein FIG. 1a is a schematic structural diagram for showing a cheek lifting method, FIG. 1b is a schematic structural diagram for showing a neck lifting method, and FIG. 1c is a schematic structural diagram for showing a forehead lifting method.

When the soft tissue of the face droops with advancing years, nasolabial skinfolds and jowl skinfolds formed near the lower lip greet the eye first of all.

The nasolabial skinfolds are inclined skinfolds formed from the sides of left nose-wing and right nose-wing toward the sides of the mouth. It has been reported that the nasolabial skinfolds are formed by various complex causes.

For instance, deep skinfolds are formed on the nasolabial portion of the face since there is no subcutaneous fat on the nasolabial portion innately, since the bone of the nasolabial portion is depressed, since the lips protrude out, or since the cheeks droop with advancing years.

However, when a patient puts both hands on whisker areas of the face and pulls up the areas, the nasolabial skinfolds and the jowl skinfolds are removed.

Using the above principle, if the fascia tissues (SMAS) of the whisker areas are pulled up strongly after the whisker areas are excised a little, the nasolabial skinfolds and the jowl skinfolds are removed as slick as nothing at all, like when the whisker areas are pulled up with both hands.

Till quite recently, the skin lifting method using the synthetic resin thread with cogs has been widely used in stead of the skin lifting surgery carried out by excising the skin, but falters a little these days since the lifting effect is not kept for a long time. In addition, since several threads are inserted under the skin and the skin is pulled up with the cogs, even though a skilled doctor performs the surgery, the patient cannot make a daily life for quite a while due to a dimple phenomenon.

However, if the lifting instrument 100 according to the present invention is used for the plastic surgery to remove facial skinfolds, the above problem can be easily solved.

As shown in FIG. 1, the lifting instrument 100 usable for removing the facial skinfolds includes: a Gore-tex thread 110 with a thickness ranging from 1.0 mm to 1.5 mm used for lifting skin tissues after being combined to the skin tissues; and needles 111 and 112 disposed at both sides of the Gore-tex thread 110 and having different curvature radiuses.

When the plastic surgery is performed for removing the facial skinfolds, the thickness and length of the Gore-tex thread 110 can be adopted properly as occasion demands. In this embodiment, the Gore-tex thread 110 has a thickness of about 1.2 mm and a length ranging from 100 cm to 120 cm.

Gore-tex, which is the main material of the Gore-tex thread 110, provides good bio-compatibility to the human body. That is, Gore-tex is not harmful to the human body since it has been widely used also as a material for implant.

Particularly, since Gore-tex has micro pores, if the cheek lifting shown in FIG. 1a, the neck lifting shown in FIG. 1b or the forehead lifting shown in FIG. 1c is performed using the Gore-tex thread 110, the Gore-tex thread 110 can be easily harmonized with the human body's tissues because the tissues of the corresponding portion are ingrown into the micro pores of the Gore-tex thread 110 while the tissues grow. Of course, the plastic surgery performed using the conventional synthetic resin thread having the thickness less than 0.5 mm cannot provide the above effect.

Meanwhile, as described above, the thread generally used in the prior arts for removing skinfolds is made of synthetic resin and has the thickness less than about 0.5 mm. So, on the assumption that the cheek lifting shown in FIG. 1a, the neck lifting shown in FIG. 1b or the forehead lifting shown in FIG. 1c is performed using the typical thread according to the prior arts, when an excessive tension or tensile force is applied to the synthetic resin thread, a sawing or eating phenomenon occurs on the skin tissues.

However, if the plastic surgery is performed using the Gore-tex thread 110 having the thickness ranging from 1.0 mm to 1.5 mm, the sawing or eating phenomenon of the skin tissues does not occur. In addition, it has been verified that, even though a Gore-tex thread with the thickness more than 2 mm is used, it is naturally positioned and fixed inside the skin tissues without being felt.

For a removal of the facial skinfolds, the needles 111 and 112 are disposed at both ends of the Gore-tex thread 110.

Of course, the needles 111 and 112 are provided for convenience in surgery, and so, removed after the surgery. As shown in FIG. 1, when the needles 111 and 112 are disposed at both ends of the Gore-tex thread 110, it generates trauma on the skin tissues less than the conventional surgery method that a thread is planted to the skin tissues by awls or spinal needles, makes the plastic surgery more convenient than the conventional surgery method, and reduces a surgery time.

The needles 111 and 112 are in the form of a fluent curve like an arrow, and have different curvature radiuses in consideration of the structure of the face. For instance, the needle 111 having a relatively smaller curvature radius has a straight line length ranging from 50 mm to 90 mm, but the needle 112 having a relatively larger curvature radius has a straight line length ranging from 40 mm to 60 mm. In this embodiment, the needle 111 located at the left in FIG. 1 is about 70 mm and the needle 112 located at the right in FIG. 1 is about 42 mm. Particularly, the needle 112 located at the right is manufactured to have a curvature radius of about ⅜ circle.

As shown in FIG. 1, now, the cheek lifting method of FIG. 1a, the neck lifting method of FIG. 1b and the forehead lifting method of FIG. 1c performed using the lifting instrument 100 for removing the facial skinfolds according to the present invention will be described.

To perform the cheek lifting, first, a doctor prepares the lifting instrument 100 shown in FIG. 1, and then, makes a temporal excision 115 at a target region of the face. The temporal excision 115 may be made at any region near to the cheek. In this embodiment, a fine temporal excision 115 is formed near the temple.

In this instance, the temporal excision 115 is vertically made to the horny integument, the multilayered epidermis and the corium layer under the epidermis at the target region of the face.

After formation of the temporal excision 115, an anchor P1, which becomes a standard of tension, is planted inside the temporal excision 115. Ends of the Gore-tex threads 110 are respectively fixed to the tissues of the nasolabial region (A), the jowl region (B) located at the side of the lips and the mandible region (C) out of heavy cheek tissues (in this instance, additional anchors may be planted in the above regions) , and the other ends of the Gore-tex threads 110 are respectively hung, pulled and fixed on the anchor P1. In this instance, since the threads must be pulled from the three directions, namely, from the nasolabial region (A), the jowl region (B) and the mandible region (C) toward the anchor P1, three Gore-tex threads 110 are separately used in the three directions.

Of course, as shown in the drawings, it is more effective that the Gore-tex threads 110 are fixed not in a single-line type but in a two-line type to transmit a more stable power. That is, the Gore-tex thread 110 is put around in a form proper to the target region and fixed in such a way that both ends meet with each other or are adjacent to each other, so that the entire form of the Gore-tex thread 110 is constructed not in the single-line type but in the two-line type where the both ends of the thread 110 are connected with each other.

As described above, since the Gore-tex thread 110 is constructed not in the single-line type but in the two-line type, power is applied to both sides uniformly when the thread is pulled to thereby prevent that the thread is broken later due to an excessive extension at one side or that an unsatisfied result is obtained due to lengthening of a unextended side. Particularly, like this embodiment, the two-line type thread provides a merit that power is transmitted uniformly when the tissues are pulled from the three directions for the cheek lifting. Of course, differently from the drawings, the Gore-tex thread 110 is formed in the two-line type, and two strands of the tread can cross each other at the center thereof once or more.

As shown in FIG. 1a, since the Gore-tex threads 110 pull the tissues from the three directions of the nasolabial region (A), the jowl region (B) and the mandible region (C) toward the anchor P1, the cheek lifting is realized to remove cheek skinfolds.

Next, the neck lifting will be described. In fact, the neck lifting method shown in FIG. 1b has been rarely developed till quite recently. Botox or filler may remove neck skinfolds temporarily, but it is not effective. The neck is a body region which is frequently moved, and so, it is difficult to perform skin excision to the neck region due to a large scar.

However, as shown in FIG. 1b, an anchor P3 is formed at a hard portion (G) of the posterior hair line, and then, an end of the Gore-tex thread 110 is hung on the tissues (F) around the SCM muscular fascia and the other end is hung on the anchor P3 and pulled, so that the tissues (F) around the SCM muscular fascia are pulled up toward a hard part (G) of the posterior hair line, whereby neck skinfolds are removed. If the neck lifting is performed through the above method, it can provide a semi-permanent effect with little influence even though the patient frequently moves the neck.

Of course, to form the anchor P3 at the hard portion of the posterior hair line, the temporal excision described above referring to FIG. 1a must be formed at the hard portion, and the Gore-tex thread 110 must be used in the two-line type and cross-arranged, but since it is described above referring to FIG. 1a, its description will be omitted to avoid repeated description.

Next, the forehead lifting will be described. The typical forehead lifting surgery to remove forehead skinfolds is performed by the steps of excising the scalp where hairs grow, pulling the forehead skin, and suturing the excised portion.

However, such a surgery method has several problems in that hairs do not grow in an area ranging 1 cm around the excised portion after the surgery and skinfolds recur before long. Recently, as a non-invasive surgery, a thread with cogs is used, but its effect is not kept for a long time.

However, the lifting instrument 100 according to the present invention shown in FIG. 1 can solve the above problems. That is, as shown in FIG. 1c, some of the scalp is excised, and then, an anchor P4 is formed at the excision. After that, an end of the Gore-tex thread 110 is hung the tissues within an eyebrow area 3 and the other end of the Gore-tex thread 110 is hung on the anchor P4 and pulled, so that the tissues within the eyebrow area 3 are pulled up toward the anchor P4, whereby the forehead skinfolds are removed.

Particularly, through the above surgery, in case of the patient who has slanted eyebrows, the eyebrows 3 can be surely lifted, and so, hidden double eyelids are exposed without blepharoplasty to thereby provide a fresh figure.

As described above, using the lifting instrument 100 shown in FIG. 1, the cheek lifting of FIG. 1a, the neck lifting of FIG. 1b and the forehead lifting of FIG. 1c can be performed easily and simply. The lifting surgery performed using the lifting instrument 100 can provide the same surgery result without regard to the doctor experiences or skills so that the doctor and the patient are all satisfied with the surgery result, keep the satisfactory surgery result longer than the prior arts, and guarantee a rapid recovery after the surgery so that the patient can rapidly return to daily life.

In addition, if the lifted cheek, neck or forehead is relaxed as time goes by, skinfolds formed at the relaxed portion can be removed again by pulling a little the Gore-tex thread 110 fixed on the anchor and fixing it again, and so, the lifting method using the lifting instrument 100 according to the present invention can provide an after service.

Removal of Body Skinfolds

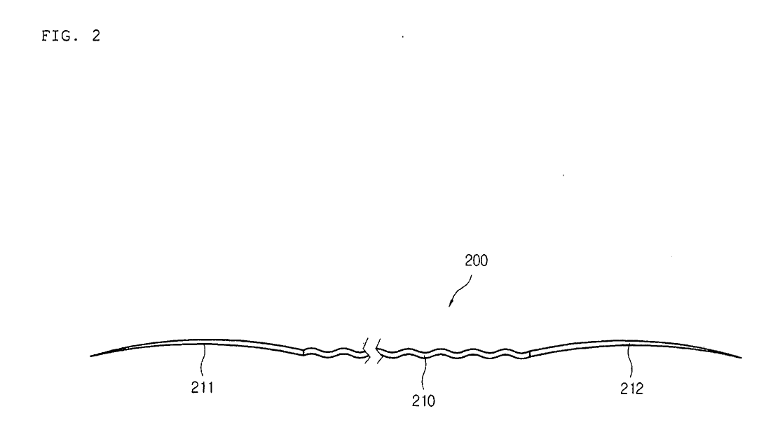

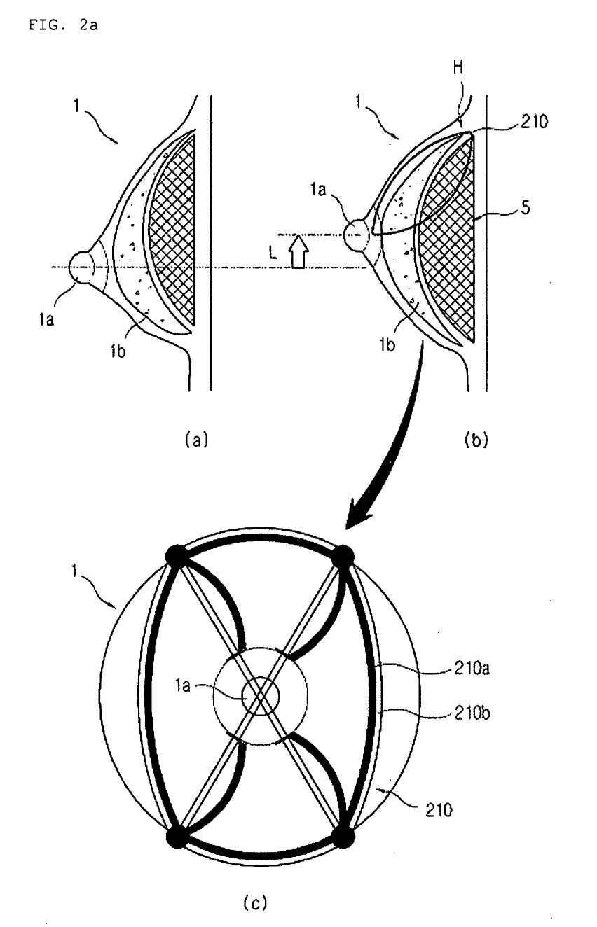

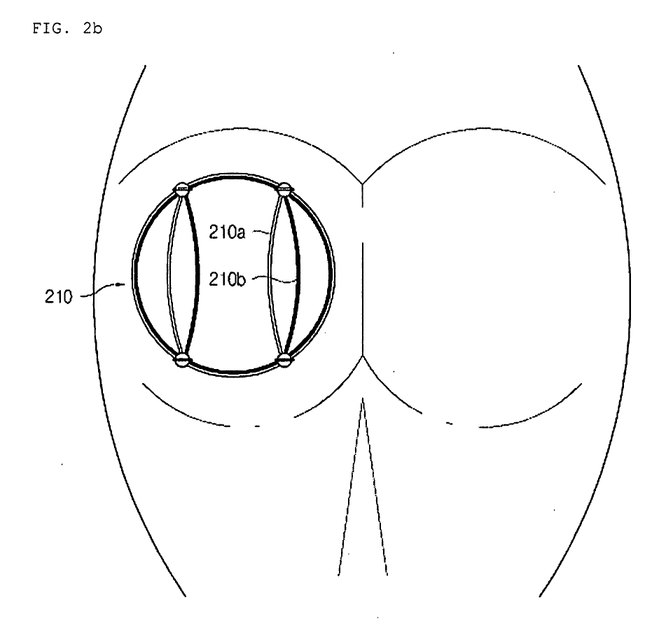

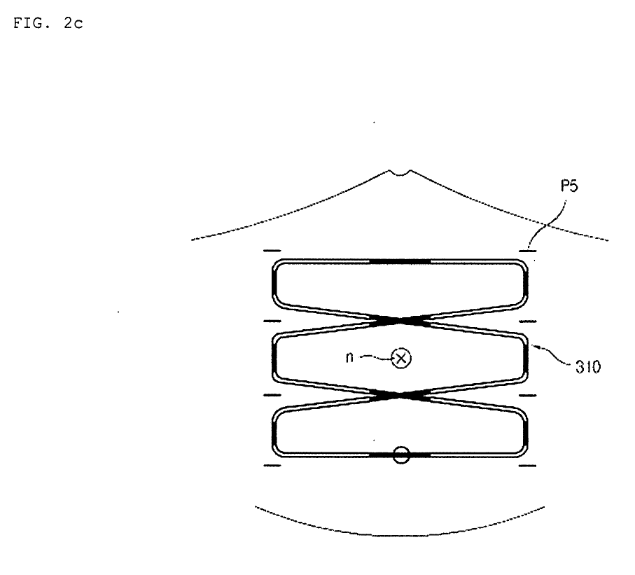

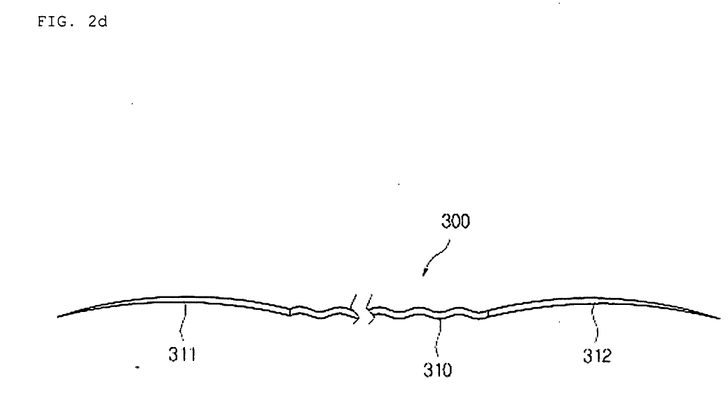

FIG. 2 is a view of a lifting instrument for removing body skinfolds according to a second preferred embodiment of the present invention, wherein FIG. 2a is a schematic structural diagram for showing a breast lifting method, FIG. 2b is a schematic structural diagram for showing a hip lifting method, FIG. 2c is a schematic structural diagram for showing an abdomen lifting method, and FIG. 2d is a view of a lifting instrument for removing abdomen skinfolds for the abdomen lifting.

As shown in FIG. 2, the lifting instrument 200 for removing body skinfolds is nearly similar with the lifting instrument 100 (see FIG. 1) for removing the facial skinfolds.

That is, the lifting instrument 200 usable for removing the body skinfolds includes: a Gore-tex thread 210 with a thickness ranging from 1.2 mm to 1.7 mm used for lifting an appropriate skin tissue after being combined to the skin tissue; and needles 211 and 212 disposed at both sides of the Gore-tex thread 210 and having different curvature radiuses.

The length of the Gore-tex thread 210 can be adopted properly as occasion demands, and in this embodiment, the Gore-tex thread 210 has a thickness of about 1.4 mm and a length ranging from 80 cm to 120 cm. The needles 211 and 212 disposed at both sides of the Gore-tex thread 210 all have a straight line length of about 120 cm.

As shown in FIGS. 2a to 2d, using the lifting instrument 200 of FIG. 2, the breast lifting, the hip lifting and the abdomen lifting can be performed.

For your reference, the Gore-tex thread 210 used for the breast lifting, the hip lifting and the abdomen lifting is different from the Gore-tex thread 110 used for the face plastic surgery in thickness and length, but has the same physical properties, structure and functions as the Gore-tex thread 110 used for the face plastic surgery. So, in this embodiment, a repeated description of the Gore-tex thread 210 will be omitted.

To perform the breast lifting, first, the lifting instrument 200 shown in FIG. 2 is prepared. As shown in (a) to (c) of FIG. 2a, an anchor (not shown) is formed at the pectoralis fascia(H).

Moreover, using the lifting instrument 200 of FIG. 2, an end of the Gore-tex thread 210 is hung on a drooped glandular tissue and the other end is hung on the anchor formed at the pectoralis fascia (H) and pulled, so that the breast lifting is performed easily and simply.

To hang the other end of the Gore-tex thread 210 on the anchor of the pectoralis fascia, as shown in (c) of FIG. 2a, a thread 210a passes through the glandular tissue 1b and the other thread 210b passes through the subcutaneous tissue, surrounds the glandular tissue 1b, and is bound thereto and fixed to the pectoralis fascia (H) to obtain a good result. So, the papilla 1a can be lifted easily and simply. In fact, if the surgery is performed as shown in (b) of FIG. 2a, the papilla 1a can rise to a height (L).

Next, the hip lifting will be described. As shown in FIG. 2b, an anchor (not shown) is formed near a hard membrane adjacent to the edge of the pelvic bone above the hip. After that, using the lifting instrument 200 of FIG. 2, an end of the Gore-tex thread 210 is hung on a drooped hip tissue and the other end is hung on the anchor and pulled, whereby the hip lifting can be performed easily and simply.

In this instance, like the breast lifting, a thread 210a may pass through the subcutaneous tissue and the other thread 210b may go near to the fascia long and be firmly fixed to the hard membrane near the edge of the pelvic bone. In addition, if the thread goes round to collect the hip tissues to the center, the hip can be up easily in a convex form.

Next, the abdomen lifting will be described. FIG. 2c is a schematic structural diagram for showing the abdomen lifting method, and FIG. 2d is a view of a lifting instrument for removing abdomen skinfolds for the abdomen lifting.

For the abdomen lifting, the lifting instrument 200 shown in FIG. 2 may be used, but the lifting instrument 300 shown in FIG. 2d is more convenient. Of course, the lifting instrument 300 for lifting the abdomen is nearly similar with the body lifting instrument 200 (see FIG. 2) in substance.

That is, the lifting instrument 300 for abdomen lifting includes a Gore-tex thread 310 with a thickness ranging from 1.8 mm to 2.3 mm used for lifting an appropriate skin tissue after being combined to the skin tissue and needles 311 and 312 disposed at both sides of the Gore-tex thread 310 and having different curvature radiuses.

The length of the Gore-tex thread 310 can be adopted properly as occasion demands, and in this embodiment, the Gore-tex thread 310 is about 2.0 mm in thickness and about 120 cm in length.

The reason is that the abdomen requires stronger pulling power than other body regions. The needles 311 and 312 disposed at both sides of the Gore-tex thread 210 all have a straight line length of about 100 mm.

To perform the abdomen lifting, first, anchors P5 are form on a hard fascia layer vertically formed at a region where the rectus abdominis and an abdominal muscle meet with each other. As repeatedly described above, the portion must be partially excised to form the anchors P5.

After formation of the anchors P5, using the lifting instrument 200 of FIG. 2, an end of the Gore-tex thread 310 is hung on the drooped abdominal tissue and the other end is hung on the anchor P5 and pulled, whereby the abdomen lifting can be performed easily and simply.

In this instance, as shown in FIG. 2c, it is preferable that the Gore-tex thread 310 is arranged in a zigzag form and simultaneously connects and collects the upper abdomen and the lower abdomen in relation with the navel (n) at a time. If the patient has much fat on the abdomen, the abdomen lifting surgery can be performed after liposuction is first performed to make the skin and the rectus abdominis closer.

Particularly, it is preferable that the Gore-tex thread 310 is arranged not in a single-line type but in a two-line type to transmit power stably. That is, the Gore-tex thread 310 is put around in a form proper to the target region and fixed in such a way that both ends meet with each other or are adjacent to each other, so that the entire form of the Gore-tex thread 310 is not in the single-line type but in the two-line type where the both ends of the thread 310 are connected with each other.

As described above, since the Gore-tex thread 310 is constructed not in the single-line type but in the two-line type, power is applied to both sides uniformly when the thread is pulled to thereby prevent that the thread is broken later due to an excessive extension at one side or that an unsatisfied result is obtained due to lengthening of a unextended side. Particularly, like this embodiment, in case where stronger power to pull the tissues is required for the abdomen lifting, the Gore-tex thread arranged in the two-line type can transmit power uniformly.

As described above, using the lifting instrument 200 of FIG. 2, the body lifting, especially, the breast lifting of FIG. 2a, the hip lifting of FIG. 2b and the abdomen lifting of FIG. 2c can be performed easily and simply. The lifting surgery performed using the lifting instrument 200 can provide the same surgery result without regard to the doctor's experiences or skills so that the doctor and the patient are all satisfied with the surgery result, keep the satisfactory surgery result longer than the prior arts, and guarantee a rapid recovery after the surgery so that the patient can rapidly return to daily life.

In addition, if the lifted breast, hip or abdomen is relaxed as time goes by, skinfolds formed at the relaxed portion can be removed again by pulling a little the Gore-tex thread 210 or 310 fixed on the anchor and fixing it again, and so, the lifting method using the lifting instrument 200 according to the present invention can provide an after service.

Reduction of Vagina

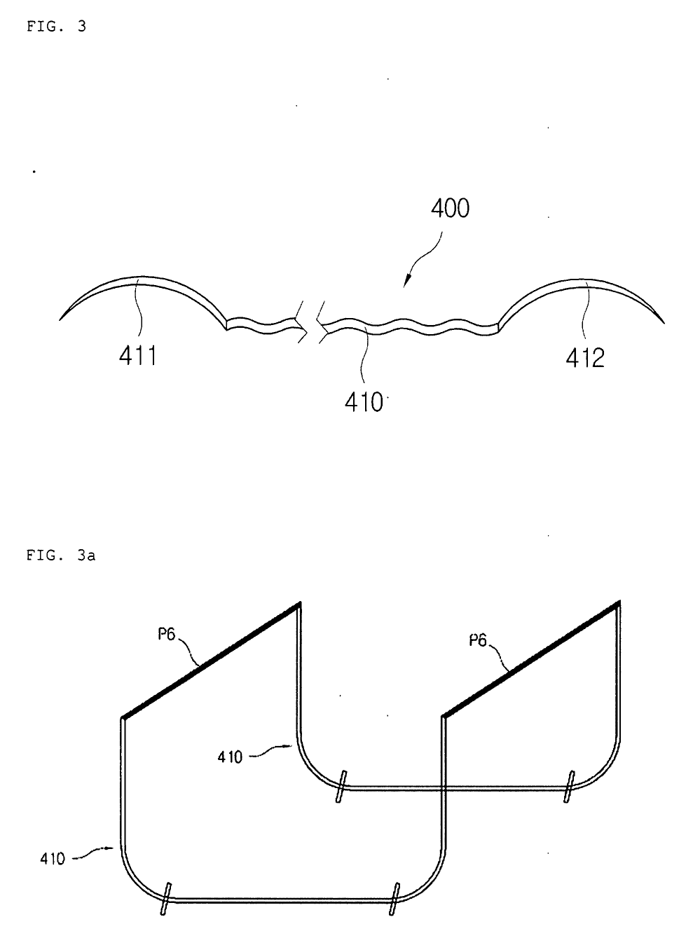

FIG. 3 is a view of a lifting instrument for removing vagina rugae according to a second preferred embodiment of the present invention, wherein, wherein FIG. 3a is a schematic structural diagram for showing a vagina lifting method.

As shown in FIG. 3, the lifting instrument 400 for removing vagina rugae, in other words, the lifting instrument 400 for reducing the vagina, is also nearly similar with the lifting instrument 100 for removing facial skinfolds (see FIG. 1) and the lifting instrument 200 for removing body skinfolds (see FIG. 2). That is, the lifting instrument 400 for vagina lifting includes: a Gore-tex thread 410 with a thickness ranging from 1.0 mm to 1.5 mm used for lifting an appropriate skin tissue after being combined to the skin tissue; and needles 411 and 412 disposed at both sides of the Gore-tex thread 410 and having different curvature radiuses.

The length of the Gore-tex thread 410 can be adopted properly as occasion demands, and in this embodiment, the Gore-tex thread 410 has a thickness of about 1.2 mm and a length of about. In addition, the needles 411 and 412 disposed at both sides of the Gore-tex thread 410 all have a length of 42 mm.

To perform the vagina reducing surgery, as shown in FIG. 3a, using the lifting instrument 400 of FIG. 3, anchors P6 are formed on the vaginal lateral wall. An end of the Gore-tex thread 410 is hung on the vaginal inferior wall and the other end is hung on the anchor P6 and pulled tightly, whereby the vaginal orifice can be reduced easily and simply. In this instance, as shown in FIG. 3a, it is preferable that the Gore-tex threads 410 are arranged symmetrically to each other along the circumference of the vaginal orifice.

As described above, using the lifting instrument 400 of FIG. 3, the vagina reducing surgery can be performed easily and simply. The lifting surgery performed using the lifting instrument 400 can provide the same surgery result for each surgery regardless of the doctor's experiences or skills so that the doctor and the patient are all satisfied with the surgery result, keep the satisfactory surgery result longer than the prior arts, and ensure that the patient rapidly returns to his or her normal life with a rapid recovery after the surgery.

Therefore, according to the present invention, the lifting instrument for removing skinfolds and method of removing skinfolds using the same can be applied to any body region through an easy and simple surgery, can achieve the same surgery result for each surgery regardless of a doctor's experiences or skills so that the doctor and a patient are all satisfied with the surgery result, can keep the satisfactory surgery result longer than the prior arts, and can ensure that the patient rapidly returns to his or her normal life with a rapid recovery after the surgery.

While the present invention has been described with reference to the particular illustrative embodiment, it is not to be restricted by the embodiment but only by the appended claims. It is to be appreciated that those skilled in the art can change or modify the embodiment without departing from the scope and spirit of the present invention.

What is claimed is:

1. A lifting instrument for removing skinfolds comprising:
a Gore-tex thread with a thickness ranging from 1.0 mm to 2.3 mm used for substantially lifting corresponding skin tissues after being combined to the skin tissues; and
needles disposed on at least one of both ends of the Gore-tex thread, the needle having an arrow-like fluent curve and a straight line length ranging from 40 mm to 140 mm.

2. The lifting instrument for removing skinfolds according to claim 1, wherein the needles are disposed at both sides of the Gore-tex thread and have different curvature radiuses.

3. The lifting instrument for removing skinfolds according to claim 1, wherein the needles are disposed at both sides of the Gore-tex thread and have the same curvature radius.

4. The lifting instrument for removing skinfolds according to claim 1 or 2, wherein in case where the lifting instrument is used for removing facial skinfolds, the Gore-tex thread has a thickness ranging from 1.0 mm to 1.5 mm, and one of the needles located at both sides of the Gore-tex thread has a straight line length ranging from 50 mm to 90 mm and the other one has a straight line length ranging from 40 mm to 60 mm.

5. The lifting instrument for removing skinfolds according to claim 1 or 3, wherein in case where the lifting instrument is used for removing body skinfolds, the Gore-tex thread has a thickness ranging from 1.2 mm to 1.7 mm, and the needles located at both sides of the Gore-tex thread have a straight line length ranging from 100 mm to 140 mm.

6. The lifting instrument for removing skinfolds according to claim 1 or 3, wherein in case where the lifting instrument is used for an abdomen lifting, the Gore-tex thread has a thickness ranging from 1.8 mm to 2.3 mm, and the needles located at both sides of the Gore-tex thread have a straight line length ranging from 80 mm to 120 mm.

7. The lifting instrument for removing skinfolds according to claim 1 or 3, wherein in case where the lifting instrument is used for a vagina lifting, the Gore-tex thread has a thickness ranging from 1.0 mm to 1.5 mm, and the needles located at both sides of the Gore-tex thread have a straight line length ranging from 40 mm to 60 mm.

8. A plasty method of removing skinfolds using a lifting instrument, the method comprising the steps of:
vertically forming a temporal excision at the target region, where skinfolds will be removed, to the horny integument, the multilayered epidermis and the corium layer under the epidermis;
fixing an anchor at the temporal excision to form a fiducial point of tension; and
hanging an end of the Gore-tex thread, which has a thickness ranging from 1.0 mm to 2.3 mm, on the anchor, and leading the other end of the Gore-tex thread to the target region through the corium under the epidermis, fixing the end of the thread to the fascia tissue of the drooped skin, and pulling the fixed thread end tightly.

9. The plasty method of removing skinfolds according to claim 8, wherein the threads are arranged symmetrically in two lines.

10. The plasty method of removing skinfolds according to claim 8, wherein the threads are cross-arranged by two lines.

11. The plasty method of removing skinfolds according to claim 8, wherein when a plastic surgery for removing cheek skinfolds is performed, the Gore-tex threads are pulled from three directions of the nasolabial region (A), the jowl region (B) and the mandible region (C) out of heavy cheek tissues.

12. The plasty method of removing skinfolds according to claim 8, wherein when a plastic surgery for removing neck skinfolds is performed, an anchor (P3) is formed at a hard portion (G) of the posterior hair line by pulling the tissues around the SCM muscular fascia using the Gore-tex thread.

13. The plasty method of removing skinfolds according to claim 8, wherein when a plastic surgery for removing breast skinfolds is performed, one thread passes through the glandular tissue and the other thread passes through the subcuraneous tissue, and then, they surround the glandular tissue and are bound with each other and fixed to the pectoralis fascia (H).

14. The plasty method of removing skinfolds according to claim 8, wherein when a plastic surgery for removing abdomen skinfolds is performed, a plurality anchors (P5) are formed on a hard fascia layer vertically formed at a region where the rectus abdominis and an abdominal muscle meet with each other, the anchors (P5) being arranged in two vertical rows and keeping predetermined intervals among lateral rows, and the Gore-tex threads are hung on the anchors sequentially in a zigzag form, and then, simultaneously connect and pull the upper abdomen and the lower abdomen to reduce an interval between the upper abdomen and the lower abdomen.

15. The plasty method of removing skinfolds according to claim 8, wherein when a plastic surgery for removing hip skinfolds is performed, one thread passes through the subcutaneous tissue and the other thread goes near to the fascia long and is firmly fixed to a hard membrane near the edge of the pelvic bone, and then, they go round to collect the hip tissues to the center, whereby the hip can be up easily in a convex form.

16. The plasty method of removing skinfolds according to claim 8, wherein when a plastic surgery for reducing the vagina is performed, anchors (P6) are formed on the vaginal lateral wall, and two Gore-tex threads, which are arranged at a predetermined interval, are pulled to lift up the vaginal inferior wall, whereby the vaginal orifice can be reduced.
